(19) United States
(12) Patent Application Publication
(10) Pub. No.: US 2010/0151034 A1
(43) Pub. Date: Jun. 17, 2010

(54) GRANULAR PHARMACEUTICAL COMPOSITION OF ATORVASTATIN FOR ORAL ADMINISTRATION

(75) Inventors: Takeshi Yano, Tokyo (JP); Soichiro Nakamura, Tokyo (JP); Hiroaki Tasaki, Chuo-ku (JP); Kazuhiro Sako, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Astellas Pharma Inc., Tokyo (JP)

(21) Appl. No.: 12/568,278

(22) Filed: Sep. 28, 2009

Related U.S. Application Data
(60) Provisional application No. 61/101,349, filed on Sep. 30, 2008.

(51) Int. Cl. A61K 31/40; A61K 9/16; A61P 19/10; A61P 25/28; A61P 13/08; A61P 3/06

(52) U.S. Cl. 424/495; 514/423; 424/494

(57) ABSTRACT

A granular pharmaceutical composition for oral administration, comprising (1) atorvastatin or a pharmaceutically acceptable salt thereof, (2) a surfactant selected from the group consisting of sodium laurylsulfate and polyoxyethylene hydrogenated castor oil, and (3) a water-soluble polymer, is disclosed.

### TECHNICAL FIELD

The present invention relates to a granular pharmaceutical composition for oral administration containing atorvastatin or a pharmaceutically acceptable salt thereof. More particularly, the present invention relates to a granular pharmaceutical composition for oral administration, comprising atorvastatin or a pharmaceutically acceptable salt thereof, a surfactant selected from the group consisting of sodium laurylsulfate and polyoxyethylene hydrogenated castor oil, and a water-soluble polymer.

### BACKGROUND ART

A conversion of 3-hydroxy-3-methylglutaryl-coenzyme A (HMG-CoA) to mevalonate is an early rate-limiting step in the biosynthetic pathway of cholesterol. This step is catalyzed by HMG-CoA reductase. Statins inhibit HMG-CoA reductase from catalyzing the conversion. Therefore, statins are generally potent lipid-lowering medications.

Atorvastatin calcium hydrate is placed on the market as Lipitor (registered trademark). This compound has a chemical name of [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate, and a structure of the formula:

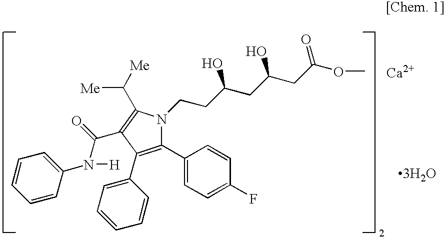

Atorvastatin and a pharmaceutically acceptable salt thereof are selective and competitive inhibitors of HMG-CoA reductase. Atorvastatin calcium is a potent lipid-lowering compound, and thus, is useful as a lipid-lowering medication and/or a cholesterol-lowering medication, and useful in treating osteoporosis, benign prostatic hypertrophy (BPH), and Alzheimer disease.

It is necessary to produce atorvastatin or a pharmaceutically acceptable salt thereof in a pure crystalline form in order to conform to a formulation containing atorvastatin or a pharmaceutically acceptable salt thereof to strict pharmaceutical requirements and specifications. Further, a process for manufacturing atorvastatin or a pharmaceutically acceptable salt thereof should be adapted to a large-scale production. Furthermore, it is preferable that the product is in a form which may be rapidly filtered and easily dried. Finally, it is economically preferable that the product is stable for a long time without special storage conditions. Various crystalline forms of atorvastatin or a pharmaceutically acceptable salt thereof were disclosed (patent literatures 1 and 2).

Since atorvastatin is a slightly soluble drug, there are problems that the dispersibility and dissolution of atorvastatin in vitro are low, and thus the bioavailability is also low. As a method to solve these problems, a method of converting the crystal to an amorphous form is known.

For example, in order to provide an improved process for producing amorphous atorvastatin, a process for forming amorphous atorvastatin, comprising the steps of dissolving atorvastatin and optionally an excipient in a non-hydroxylic solvent to form a solution, and lyophilizing the solution to afford said amorphous atorvastatin (patent literature 3), and a process for forming amorphous atorvastatin, comprising the steps of dissolving atorvastatin in a solution comprising a hydroxylic solvent, and rapidly evaporating said hydroxylic solvent from said solution to form amorphous atorvastatin (patent literature 4) are disclosed.

Further, a solid pharmaceutical composition comprising a solid dispersion of amorphous atorvastatin and one or more optional pharmaceutically acceptable excipients, the solid dispersion comprising: amorphous atorvastatin or a pharmaceutically acceptable complex, salt, solvate or hydrate thereof; and a melt-processible polymer is disclosed in order to provide a process for producing amorphous atorvastatin, which does not involve the use of volatile organic solvents, and a stable pharmaceutical composition containing amorphous atorvastatin (patent literature 5).

Furthermore, a method in which a heat treatment is carried out at a specific temperature to stabilize amorphous atorvastatin is disclosed (patent literature 6).

Atorvastatin or a pharmaceutically acceptable salt thereof tastes extremely bitter. The drug or a composition containing the same is sometimes treated with a coating or the like which inhibits release of the drug in the buccal cavity for a certain period of time, in order to mask the unpleasant taste of the drug and avoid absorption of the drug in the buccal cavity. Because water-insoluble polymers are generally used as such a coating, there is a problem that the dispersibility and dissolution of the drug is low.

Under these circumstances, there is still room for further improvements in providing a granular pharmaceutical composition for oral administration having a rapid dispersibility or dissolution in the gastrointestinal tract, even if crystalline atorvastatin or a pharmaceutically acceptable salt thereof is used and the unpleasant taste of the drug in the buccal cavity is masked (drug dosing compliance).

### CITATION LIST

### PATENT LITERATURE

[patent literature 1] Japanese Patent No. 3296564 (types I, II, and IV)
[patent literature 2] Japanese Patent No. 3965155 (types V, VI, VII, VIII, IX, X, XI, XII, XIII, XIV, XV, XVI, XVII, XVIII, and XIX)
[patent literature 3] International Publication No. WO 2006/046109
[patent literature 4] International Publication No. WO 2004/110407
[patent literature 5] International Publication No. WO 2006/059224
[patent literature 6] International Publication No. WO 2007/034316

### SUMMARY OF INVENTION

### Technical Problem

An object of the present invention, with respect to atorvastatin having an unpleasant taste or a pharmaceutically acceptable salt thereof, is to provide a granular pharmaceutical composition which contains high density atorvastatin or a pharmaceutically acceptable salt thereof having uniform particle sizes; exhibits a rapid dispersibility or dissolution in the gastrointestinal tract, even if the unpleasant taste of the drug in the buccal cavity is masked; and has a sufficient strength so that the drug-containing particles are not easily disintegrated after coating or tabletting.

### Solution to Problem

Under these circumstances, the present inventors focused on particles in which crystalline atorvastatin or a pharmaceutically acceptable salt thereof is used, the unpleasant taste of the drug in the buccal cavity is masked, and a rapid dissolution is achieved, and conducted intensive studies, and as a result, found that a granular pharmaceutical composition for oral administration having a rapid dissolution can be provided by a combination of a specific surfactant and a water-soluble polymer.

The present invention provides:
[1] a granular pharmaceutical composition for oral administration, comprising (1) atorvastatin or a pharmaceutically acceptable salt thereof, (2) a surfactant selected from the group consisting of sodium laurylsulfate and polyoxyethylene hydrogenated castor oil, and (3) a water-soluble polymer;
[2] the granular pharmaceutical composition for oral administration of [1], wherein an amount of the surfactant is 30% by weight to 200% by weight with respect to an amount of atorvastatin or a pharmaceutically acceptable salt thereof;
[3] the granular pharmaceutical composition for oral administration of [1] or [2], wherein an amount of the surfactant is 40% by weight to 100% by weight with respect to an amount of atorvastatin or a pharmaceutically acceptable salt thereof;
[4] the granular pharmaceutical composition for oral administration of any one of [1] to [3], wherein the surfactant is sodium laurylsulfate;
[5] the granular pharmaceutical composition for oral administration of any one of [1] to [4], wherein the water-soluble polymer has a viscosity of approximately 2 mPa·s to approximately 100 mPa·s;
[6] the granular pharmaceutical composition for oral administration of any one of [1] to [5], wherein the water-soluble polymer is one member, or two or more of members selected from the group consisting of hydroxypropylmethyl cellulose, hydroxypropyl cellulose, methyl methacrylate-butyl methacrylate-dimethylaminoethyl methacrylate copolymer coexistent with an acidic substance in an amount which neutralizes 10% or more of basic groups of the copolymer, povidone, and methyl cellulose;
[7] the granular pharmaceutical composition for oral administration of any one of [1] to [6], wherein an amount of the water-soluble polymer is 5% by weight to 100% by weight with respect to an amount of atorvastatin or a pharmaceutically acceptable salt thereof;
[8] the granular pharmaceutical composition for oral administration of any one of [1] to [7], wherein an amount of the water-soluble polymer is 10% by weight to 40% by weight with respect to an amount of atorvastatin or a pharmaceutically acceptable salt thereof;
[9] the granular pharmaceutical composition for oral administration of any one of [1] to [8], which contains a core;
[10] the granular pharmaceutical composition for oral administration of [9], wherein the core is coated with a coating comprising (1) atorvastatin or a pharmaceutically acceptable salt thereof, (2) the surfactant selected from the group consisting of sodium laurylsulfate and polyoxyethylene hydrogenated castor oil, and (3) the water-soluble polymer;
[11] the granular pharmaceutical composition for oral administration of [9] or [10], wherein the core is one member, or two or more of members selected from the group consisting of crystalline cellulose, purified sucrose spheres, D-mannitol, magnesium hydroxide, lactose-crystalline cellulose spheres, and sucrose-starch spheres;
[12] the granular pharmaceutical composition for oral administration of any one of [1] to [11], wherein atorvastatin or a pharmaceutically acceptable salt thereof exists as a crystal;
[13] the granular pharmaceutical composition for oral administration of any one of [1] to [12], wherein atorvastatin or a pharmaceutically acceptable salt thereof exists as a type I crystal;
[14] the granular pharmaceutical composition for oral administration of any one of [1] to [13], wherein atorvastatin or a pharmaceutically acceptable salt thereof is atorvastatin calcium;
[15] a process of manufacturing a granular pharmaceutical composition for oral administration, comprising mixing atorvastatin or a pharmaceutically acceptable salt thereof with a water-soluble polymer and a surfactant selected from the group consisting of sodium laurylsulfate and polyoxyethylene hydrogenated castor oil;
[16] the process of [15], wherein an amount of the surfactant is 30% by weight to 200% by weight with respect to an amount of atorvastatin or a pharmaceutically acceptable salt thereof;
[17] the process of [15] or [16], wherein an amount of the surfactant is 40% by weight to 100% by weight with respect to an amount of atorvastatin or a pharmaceutically acceptable salt thereof;
[18] the process of any one of [15] to [17], wherein the surfactant is sodium laurylsulfate;
[19] the process of any one of [15] to [18], wherein the water-soluble polymer has a viscosity of approximately 2 mPa·s to approximately 100 mPa·s;
[20] the process of any one of [15] to [19], wherein the water-soluble polymer is one member, or two or more of members selected from the group consisting of hydroxypropylmethyl cellulose, hydroxypropyl cellulose, methyl methacrylate-butyl methacrylate-dimethylaminoethyl methacrylate copolymer coexistent with an acidic substance in an amount which neutralizes 10% or more of basic groups of the copolymer, povidone, and methyl cellulose;
[21] the process of any one of [15] to [20], wherein an amount of the water-soluble polymer is 5% by weight to 100% by weight with respect to an amount of atorvastatin or a pharmaceutically acceptable salt thereof;
[22] the process of any one [15] to [21], wherein an amount of the water-soluble polymer is 10% by weight to 40% by weight with respect to an amount of atorvastatin or a pharmaceutically acceptable salt thereof;
[23] the process of any one of [15] to [22], which contains a core;
[24] the process of [23], wherein the core is coated with a coating comprising (1) atorvastatin or a pharmaceutically acceptable salt thereof, (2) the surfactant selected from the group consisting of sodium laurylsulfate and polyoxyethylene hydrogenated castor oil, and (3) the water-soluble polymer;
[25] the process of [23] or [24], wherein the core is one member, or two or more of members selected from the group consisting of crystalline cellulose, purified sucrose spheres, D-mannitol, magnesium hydroxide, lactose-crystalline cellulose spheres, and sucrose-starch spheres;
[26] the process of any one of [15] to [25], wherein atorvastatin or a pharmaceutically acceptable salt thereof is atorvastatin calcium; and
[27] use of a water-soluble polymer and a surfactant selected from the group consisting of sodium laurylsulfate and polyoxyethylene hydrogenated castor oil in the manufacture of a granular pharmaceutical composition for oral administration having a rapid dissolution of atorvastatin or a pharmaceutically acceptable salt thereof in the gastrointestinal tract.

### Advantageous Effects of Invention

According to the present invention,
(1) with respect to atorvastatin having an unpleasant taste or a pharmaceutically acceptable salt thereof, a rapid dissolution in the gastrointestinal tract, even if the unpleasant taste in the buccal cavity is masked;
(2) drug dosing compliance and avoidance of a delay in absorption; and
(3) a sufficient strength so that drug-containing particles are not easily disintegrated after coating or tabletting, by using particles which contains high density atorvastatin or a pharmaceutically acceptable salt thereof having uniform particle sizes, as the drug-containing particles; can be achieved.

### DESCRIPTION OF EMBODIMENTS

Hereinafter the granular pharmaceutical composition for oral administration of the present invention will be explained.

The term “granular pharmaceutical composition” as used herein a drug-containing granular composition which has a size less than a certain value described below and may be orally administered together with one pharmaceutical additive or two or more pharmaceutical additives in various forms. In the case that the shape of the granular composition may be approximate to a sphere, the size of the granular pharmaceutical composition is defined as an average particle size of 2 mm or less. In the case that the shape of the granular pharmaceutical composition is not a sphere, the size of the granular pharmaceutical composition is defined as an average maximum length of 2 mm or less.

In this regard, the lower limit is not particularly limited, so long as it is within a pharmaceutically acceptable range. The size of the granular pharmaceutical composition is, for example, 1 μm or more, 10 μm or more in another embodiment, and 20 μm or more in still another embodiment.

The particle size may be determined by, for example, an optical microscopy method described in the General Tests section of the 15th Edition of the Japanese Pharmacopoeia. In the optical microscopy method, an optical microscopy is used to observe the morphogical appearance and shape of the individual particles either directly with the naked eye or by using a microscopic photograph, in order to measure the particle size. A triaxial average particle size or a biaxial average particle size may be used as the particle size. Alternatively, a micro compression testing machine (Shimadzu Micro Compression Testing Machine, manufactured by Shimadzu Corporation) may be used to observe the morphogical appearance and shape of the individual particles directly with the naked eye, in order to measure the particle size, and a biaxial average particle size may be used as the particle size in this method.

The term “rapid dispersibility or dissolution” as used herein means a dissolution rate determined by a dissolution test, method 2 described in the Japanese Pharmacopoeia using 900 mL of the first fluid (JP1) described in this dissolution test of the Japanese Pharmacopoeia, is 50% or more for 15 minutes, or 60% or more for 15 minutes in another embodiment.

Atorvastatin or a pharmaceutically acceptable salt thereof used in the present invention includes atorvastatin calcium hydrate disclosed in U.S. Pat. No. 5,273,995 having a chemical name of [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate. Atorvastatin calcium hydrate has a structure of the formula:

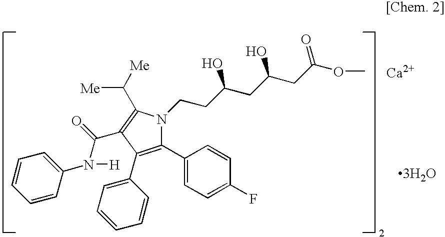

and is placed on the market as Lipitor (registered trademark). Atorvastatin or a pharmaceutically acceptable salt thereof is a selective and competitive inhibitor of HMG-CoA reductase. Examples of the pharmaceutically acceptable salt include a metal salt such as an alkaline metal salt and an alkaline earth metal salt, and an amine salt such as an organic amine. In another embodiment, examples thereof include salts with sodium, potassium, lithium, calcium, magnesium, aluminum, iron, zinc, calcium carbonate, calcium hydroxide, magnesium carbonate, magnesium hydroxide, magnesium silicate, magnesium aluminate, or aluminum magnesium hydroxide. In still another embodiment, a calcium salt may be used. Atorvastatin calcium is a potent lipid-lowering compound, and thus, is useful as a lipid-lowering medication and/or a cholesterol-lowering medication, and also useful in treating osteoporosis, benign prostatic hypertrophy (BPH), and Alzheimer disease. Examples of crystalline-form atorvastatins include types I, II, IV, V, VI, VII, VIII, IX, X, XI, XII, XIII, XIV, XV, XVI, XVII, XVIII, and XIX and, in another embodiment, type I. The term “type I crystal” as used herein means crystalline Form I atorvastatin hydrate disclosed in Japanese Patent No. 3296564.

The content of atorvastatin or a pharmaceutically acceptable salt thereof is not particularly limited, so long as it is a pharmaceutically effective amount for prevention or treatment. The drug is administered to an adult patient at, for example, a daily dose of approximately 2.5 mg to approximately 80 mg, a daily dose of approximately 5 mg to approximately 500 mg in another embodiment, a daily dose of approximately 2.5 mg to approximately 80 mg in still another embodiment, or a daily dose of approximately 0.1 mg/kg to approximately 8.0 mg/kg. A daily dose in another embodiment is within a range of approximately 0.1 mg/kg to approximately 2.0 mg/kg. The content may be changed or adjusted to 5 mg to 80 mg, or 5 mg to 100 mg in another embodiment, in accordance with the effect or application. In common treatment, a drug is administered to a patient in an amount less than the optimum dose at an early stage. The dose is gradually increased in accordance with the conditions until optimum effect is achieved. The daily dose can be divided into multiple doses per day, if necessary.

The content of the drug may be generally selected in accordance with the type of the drug, the application (indications) of the drug, or the age (or the weight) of a patient, and is not particularly limited, so long as it is a therapeutically or prophylactically effective amount. For example, the content is 0.5% by weight to 90% by weight with respect to the amount of the “granular pharmaceutical composition” or the pharmaceutical formulation of the present invention, 0.5% by weight to 80% by weight in another embodiment, and 0.5% by weight to 70% by weight in still another embodiment.

Sodium laurylsulfate or polyoxyethylene hydrogenated castor oil used in the present invention is not particularly limited, so long as it can improve the dispersibility of atorvastatin or a pharmaceutically acceptable salt thereof. Examples of sodium laurylsulfate include product names NIKKOL SLS (Nikko Chemicals Co., Ltd.), EMAL 0 (Kao Corporation), TEXAPON K12 P PH (Cognis Japan Ltd.), and TEXAPON K12 G PH (Cognis Japan Ltd.). Examples of polyoxyethylene hydrogenated castor oil include product names NIKKOL HCO-40 (Nikko Chemicals Co., Ltd.), NIKKOL HCO-60 (Nikko Chemicals Co., Ltd.), EMANON CH-40 (Kao Corporation), EMANON CH-60 (Kao Corporation), Noigen HC-400 (Dai-ichi Kogyo Seiyaku Co., Ltd.), Noigen HC-600 (Dai-ichi Kogyo Seiyaku Co., Ltd.), Uniox HC-40 (NOF Corporation), Uniox HC-60 (NOF Corporation), EUMULGIN HRE40 (Cognis Japan Ltd.), EUMULGIN HRE60 (Cognis Japan Ltd.), Cremophor CO 40 (BASF), and Cremophor CO 60 (BASF).

These surfactants may be used alone, or as an appropriate combination of two or more thereof.

The content of sodium laurylsulfate and/or polyoxyethylene hydrogenated castor oil is not particularly limited, so long as the content is pharmaceutically acceptable, and can improve the dispersibility of atorvastatin or a pharmaceutically acceptable salt thereof. The content as the total amount thereof may be, for example, 30% by weight to 200% by weight with respect to the amount of atorvastatin or a pharmaceutically acceptable salt thereof, 40% by weight to 100% by weight in another embodiment, and 60% by weight to 100% by weight with respect to the amount of atorvastatin or a pharmaceutically acceptable salt thereof in still another embodiment.

The water-soluble polymer used in the present invention is not particularly limited, so long as it can improve the dispersibility of atorvastatin or a pharmaceutically acceptable salt thereof, together with sodium laurylsulfate and/or polyoxyethylene hydrogenated castor oil. In the case that the water-soluble polymer is added, together with atorvastatin or a pharmaceutically acceptable salt thereof as well as sodium laurylsulfate and/or polyoxyethylene hydrogenated castor oil, into a pharmaceutically acceptable particle, the water-soluble polymer also functions as a binder which binds atorvastatin or a pharmaceutically acceptable salt thereof to the particle.

The viscosity of the water-soluble polymer may be preferably approximately 2 mPa·s to approximately 100 mPa·s, approximately 2 mPa·s to approximately 50 mPa·s in another embodiment, approximately 3 mPa·s to approximately 10 mPa·s in still another embodiment, as a viscosity at a polymer concentration of 2% at 20° C.

Examples of the water-soluble polymer include hydroxypropylmethyl cellulose (also known as hypromellose)(TC-5, Shin-Etsu Chemical Co., Ltd.), hydroxypropyl cellulose (NISSO HPC, Nippon Soda Co., Ltd.), methyl methacrylate-butyl methacrylate-dimethylaminoethyl methacrylate copolymer containing an acidic substance in an amount which neutralizes 10% or more of basic groups of the copolymer described in Japanese Patent No. 3563070 [aminoalkylmethacrylate copolymer E (Product name: Eudragit E100, Evonik Degussa GmbH)], povidone (Kollidon, BASF), and methyl cellulose (METOLOSE, Shin-Etsu Chemical Co., Ltd.); and, in another embodiment, hydroxypropylmethyl cellulose (also known as hypromellose), hydroxypropyl cellulose, and methyl methacrylate-butyl methacrylate-dimethylaminoethyl methacrylate copolymer containing an acidic substance in an amount which neutralizes 10% or more of basic groups of the copolymer.

These water-soluble polymers may be used alone, or as an appropriate combination of two or more thereof.

The content of the water-soluble polymer(s) is not particularly limited, so long as the content is pharmaceutically acceptable, and can improve the dispersibility of atorvastatin or a pharmaceutically acceptable salt thereof, together with sodium laurylsulfate. The content may be, for example, 5% by weight to 100% by weight with respect to the amount of atorvastatin or a pharmaceutically acceptable salt thereof, 10% by weight to 40% by weight in another embodiment, and 20% by weight to 40% by weight in still another embodiment.

The core used in the present invention is not particularly limited, so long as it can be a base capable of forming a pharmaceutical acceptable particle. The core is a base which constitutes the granular pharmaceutical composition of the present invention and is to be coated with the coating used in the present invention. The core is formed from a drug per se or pharmaceutical acceptable additives. A particle [for example, crystalline cellulose (particle) (sometimes referred to as microcrystalline cellulose), lactose-starch, or the like] may be used as the core. A drug alone, or a mixture of a drug and pharmaceutically acceptable additives may be used as the core. Known techniques may be used to prepare a particle consisting of one additive, or two or more additives in order to use the particle as the core. A particle of additives as an appropriate core may be sprayed with a solution or dispersion of a drug and a binder. The outward appearance of the particle may be in various forms such as a spherical shape or a cubic shape, so long as it is a pharmaceutically acceptable particle. Examples of the particle include lactose monohydrate, sodium chloride, crystalline cellulose [crystalline cellulose (particle), spherical core particles of crystalline cellulose] (for example, product name Celphere, Asahi Kasei Chemicals Corporation), purified sucrose spheres (for example, product name Nonpareil-103, Freund Corporation), sodium hydrogen carbonate, D-mannitol (for example, product name Nonpareil-108, Freund Corporation), magnesium hydroxide, magnesium carbonate, magnesium oxide, anhydrous dibasic calcium phosphate, magnesium aluminometasilicate, lactose-crystalline cellulose spheres (for example, product name Nonpareil-105, Freund Corporation), and sucrose-starch spheres (for example, product name Nonpareil-101, Freund Corporation); in another embodiment, crystalline cellulose, purified sucrose spheres, D-mannitol, magnesium hydroxide, lactose-crystalline cellulose spheres, and sucrose-starch spheres; and, in still another embodiment, crystalline cellulose.

The particle size of the core particle is not particularly limited, so long as it is within a pharmaceutically acceptable range. The particle size may be, for example, 1 μm to 1000 μm, 5 μm to 500 μm in another embodiment, and 10 μm to 300 μm in still another embodiment.

The core particle may be contained in an amount capable of obtaining a strength required as a particle containing atorvastatin or a pharmaceutically acceptable salt thereof. The content of the core particle is, for example, 0% by weight to 500% by weight with respect to the amount of atorvastatin or a pharmaceutically acceptable salt thereof, 20% by weight to 500% by weight with respect to the amount of atorvastatin or a pharmaceutically acceptable salt thereof in another embodiment, 0% by weight to 300% by weight in still another embodiment, and 50% by weight to 240% by weight with respect to the amount of atorvastatin or a pharmaceutically acceptable salt thereof in still another embodiment.

The granular pharmaceutical composition for oral administration of the present invention may be formulated appropriately using one or more various pharmaceutical fillers if desired. Such fillers include, for example, binders, disintegrating agents, acidulants, foaming agents, artificial sweeteners, flavors, lubricants, coloring agents, stabilizers, buffers, antioxidants, surfactants, and the like.

Examples of the binders include ethyl cellulose, sorbitol, maltose, powdered acacia, and the like.

Examples of the disintegrating agents include corn starch, potato starch, carmellose calcium, carmellose sodium, and the like.

Examples of the acidulants include citric acid, tartaric acid, malic acid, and the like.

Examples of the foaming agents include sodium bicarbonate and the like.

Examples of the artificial sweeteners include saccharin sodium, dipotassium glycyrrhizinate, aspartame, stevia, somatin, and the like.

Examples of the flavors include lemon, lemon lime, orange, menthol, and the like.

Examples of the lubricants include magnesium stearate, calcium stearate, sucrose fatty acid ester, polyethylene glycol, talc, stearic acid, and the like.

Examples of the coloring agents include yellow ferric oxide, red ferric oxide, food yellow No. 4, food yellow No. 5, food red No. 3, food red No. 102, food blue No. 3, and the like.

Examples of the buffers include citric acid, succinic acid, fumaric acid, tartaric acid, ascorbic acid, and salts thereof; glutamic acid, glutamine, glycine, aspartic acid, alanine, arginine, and salts thereof; magnesium oxide, zinc oxide, magnesium hydroxide, phosphoric acid, boric acid, and salts thereof; and the like.

Examples of the antioxidants include ascorbic acid, dibutyl hydroxytoluene, propyl gallate, and the like.

Examples of the surfactants include polysorbate 80 and the like.

These pharmaceutical fillers may be appropriately added alone, or as a combination of two or more thereof, in appropriate amount.

The content of these various pharmaceutical fillers is, for example, 0.1% by weight to 100% by weight with respect to a drug-containing particle, 0.1% by weight to 80% by weight in another embodiment, and 0.1% by weight to 50% by weight in still another embodiment.

The granular pharmaceutical composition of the present invention may be used to prepare various pharmaceutical formulations, which include, for example, powder, granules, dry syrups, tablets, rapidly disintegrating tablets in the buccal cavity, and the like.

Hereinafter the rapidly disintegrating tablet in the buccal cavity of the present invention containing the granular pharmaceutical composition of the present invention will be explained, but the pharmaceutical formulation of the present invention is not limited thereto.

The term “rapidly disintegrating tablet in the buccal cavity” as used herein means a tablet (or other formulations similar to a tablet) which is disintegrated in the buccal cavity within 2 minutes, 1 minute in another embodiment, and 45 seconds in still another embodiment, by substantially saliva only, without taking water for swallowing the tablets.

The granular pharmaceutical composition of the present invention may be contained in a rapidly disintegrating tablet in the buccal cavity. The granular pharmaceutical composition of the present invention may be used as a drug contained in known rapidly disintegrating tablets in the buccal cavity, as described in WO 95/20380 (corresponding U.S. Pat. No. 5,576,014), WO 2002/92057 (corresponding US Patent Application Publication No. 2003/099701), U.S. Pat. No. 4,305,502, U.S. Pat. No. 4,371,516, Japanese Patent No. 2807346 (corresponding U.S. Pat. No. 5,466,464), Japanese Unexamined Patent Publication (kokai) No. 5-271054 (corresponding EP Patent No. 553777), Japanese Unexamined Patent Publication (kokai) No. 10-182436 (corresponding U.S. Pat. No. 5,958,453), Japanese Patent No. 3412694 (corresponding U.S. Pat. No. 5,223,264), WO 98/02185 (corresponding U.S. Pat. No. 6,287,596), and WO 2008/032767 (corresponding US Patent Application Publication No. 2008/0085309), and a base for rapidly disintegrating tablets in the buccal cavity, as described in these documents, may be used to prepare a rapidly disintegrating tablet in the buccal cavity in accordance with a method as described in these documents. As described above, examples of rapidly disintegrating tablets in the buccal cavity containing a granular pharmaceutical composition include rapidly disintegrating tablets in the buccal cavity described in Japanese Patent No. 3412694 (corresponding U.S. Pat. No. 5,223,264) and Japanese Unexamined Patent Publication (kokai) No. 2003-55197, and the granular pharmaceutical composition of the present invention may be contained in these rapidly disintegrating tablets in the buccal cavity.

In general, rapidly disintegrating tablets in the buccal cavity are mainly classified into a molding type, a wet type, and a conventional tabletting type. The granular pharmaceutical composition of the present invention may be contained in any type of these rapidly disintegrating tablets in the buccal cavity. The molding type is the one prepared by filling a solution or suspension containing a filler or the like in a mold, and drying it, as disclosed in, for example, Japanese Patent No. 2807346 (corresponding to U.S. Pat. No. 5,466,464). The molding type of rapidly disintegrating tablet in the buccal cavity containing the granular pharmaceutical composition of the present invention may be prepared, for example, by filling a solution or suspension containing the granular pharmaceutical composition of the present invention, a filler such as a saccharide, and a binder such as gelatin or agar into a PTP pocket, and removing water therefrom by lyophilization, drying under reduced pressure, low-temperature drying, or the like. The wet type is the one prepared by moistening a filler such as a saccharide, tabletting it at a low pressure, and drying the tablet, as disclosed in, for example, Japanese Patent No. 3069458 (corresponding to U.S. Pat. No. 5,501,861 and U.S. Pat. No. 5,720,974). The wet type may be prepared, for example, by moistening the granular pharmaceutical composition of the present invention and a filler such as a saccharide with a small amount of water or a mixture of water and alcohol, tabletting the wet mixture at a low pressure, and drying it.

The conventional tabletting type is the one prepared by carrying out a conventional tabletting step, as disclosed in WO 95/20380 (corresponding to U.S. Pat. No. 5,576,014), WO 2002/92057 (corresponding US Patent Application Publication No. 2003/099701), Japanese Unexamined Patent Publication (kokai) No. 10-182436 (corresponding to U.S. Pat. No. 5,958,453), Japanese Unexamined Patent Publication (kokai) No. 9-48726, Japanese Unexamined Patent Publication (kokai) No. 8-19589 (corresponding to U.S. Pat. No. 5,672,364), Japanese Patent No. 2919771, Japanese Patent No. 3069458 (corresponding to U.S. Pat. No. 5,501,861, and corresponding to U.S. Pat. No. 5,720,974), and WO 2008/032767 (corresponding US Patent Application Publication No. 2008/0085309). The conventional tabletting type of rapidly disintegrating tablet in the buccal cavity containing the granular pharmaceutical composition of the present invention may be prepared by granulating the granular pharmaceutical composition of the present invention and a filler such as a saccharide having a low moldability with a solution or suspension containing a saccharide having a high moldability or a water-soluble polymer, and compression-molding granules into a compression-molded product, and optionally further drying the compression-molded product under a humidity condition, as disclosed in, for example, WO 95/20380 (corresponding to U.S. Pat. No. 5,576,014) and Japanese Patent No. 2919771. A conventional tabletting type of rapidly disintegrating tablet in the buccal cavity as disclosed in WO 99/47124 (corresponding to U.S. Pat. No. 6,589,554) may be prepared, for example, by compression-molding the granular pharmaceutical composition of the present invention and a filler such as a crystalline saccharide using an amorphous saccharide, and drying it under a humidity condition. A conventional tabletting type of rapidly disintegrating tablet in the buccal cavity as disclosed in WO 2002/92057 (corresponding US Patent Application Publication No. 2003/099701) may be prepared, for example, by compression-molding a mixture of the granular pharmaceutical composition of the present invention and a filler with a saccharide having a melting point lower than that of the filler, and heating the compression-molded product to form a crosslinkage with a melted and solidified saccharide having a low melting point. These treatments such as drying under a humidity condition or heating can improve the tablet strength of the rapidly disintegrating tablet in the buccal cavity. A conventional tabletting type of rapidly disintegrating tablet in the buccal cavity as disclosed in WO 2008/032767 (corresponding US Patent Application Publication No. 2008/0085309) may be prepared, for example, by compression-molding a mixture of the granular pharmaceutical composition of the present invention and a filler with a treated starch having a degree of gelatinization of 30% to 60%.

As fillers used in the rapidly disintegrating tablet in the buccal cavity of the present invention, conventional fillers may be used, and pharmaceutically acceptable saccharides are preferable. More particularly, a saccharide having a low moldability may be used with respect to techniques utilizing moldability of saccharides, a crystalline saccharide may be used with respect to techniques for improving a tablet strength by crystalline/amorphous properties of saccharides and drying under a humidity condition, and a saccharide having a high melting point as well as conventional fillers may be used with respect to a crosslinking technique using a melted and solidified saccharide.

The term “saccharide having a low moldability” as used herein means that, for example, when 150 mg of saccharide is formed into tablets using a punch of 8 mm in diameter under a tabletting pressure of 10 kg/cm² to 50 kg/cm², the tablets show a hardness of 0 kp to 2 kp. The term “saccharide having a high moldability” as used herein means that the tablets show a hardness of 2 kp or more, under the same conditions. Examples of saccharides having a low moldability, which are pharmaceutically acceptable, include lactose, mannitol, glucose, sucrose, xylitol, and erythritol. These saccharides may be used alone, or as an appropriate combination of two or more thereof. Examples of saccharides having a high moldability, which are pharmaceutically acceptable, include maltose, maltitol, sorbitol, and trehalose. These saccharides also may be used alone, or as an appropriate combination of two or more thereof.

Examples of the “crystalline saccharide”, which is pharmaceutically acceptable, include mannitol, maltitol, erythritol, and xylitol. These saccharides may be used alone, or as an appropriate combination of two or more thereof. Examples of the “amorphous saccharide”, which is pharmaceutically acceptable, include lactose, sucrose, glucose, sorbitol, maltose, and trehalose. These saccharides also may be used alone, or as an appropriate combination of two or more thereof.

Examples of the “saccharide having a high melting point”, which is pharmaceutically acceptable, include xylitol, trehalose, maltose, sorbitol, erythritol, glucose, sucrose, maltitol, and mannitol. These saccharides may be used alone, or as an appropriate combination of two or more thereof. Examples of the “saccharide having a low melting point”, which is pharmaceutically acceptable, include xylitol, trehalose, maltose, sorbitol, erythritol, glucose, sucrose, maltitol, and mannitol. These saccharides also may be used alone, or as an appropriate combination of two or more thereof. Examples of a binder for rapidly disintegrating tablets in the buccal cavity include maltitol and copolyvidone. These binders may be used alone, or as an appropriate combination of two or more thereof.

When a water-soluble polymer is used instead of the saccharide having a high moldability, for example, hydroxypropyl cellulose, hydroxypropylmethyl cellulose, povidone, polyvinyl alcohol, powdered acacia, gelatin, pullulan, and the like are preferable.

The term “gelatinization” as used herein means a swelling caused by physically treating a starch, thereby introducing water into the space between starch molecules. An increasing degree of gelatinization means the progress of gelatinization. Examples of a treated starch include corn starch, wheat starch, potato starch, rice starch, and cassava starch.

The content of fillers used in the rapidly disintegrating tablet in the buccal cavity containing the granular pharmaceutical composition of the present invention, or the total amount of fillers contained in the formulation may be appropriately adjusted in accordance with the content of the granular pharmaceutical composition of the present invention, the size of the tablet, and/or the like, and it is preferably 20 mg to 1000 mg per tablet in general, 50 mg to 900 mg in another embodiment, and 50 mg to 800 mg in still another embodiment.

The contents of the saccharide having a high moldability, the water-soluble polymer, the amorphous saccharide, and the saccharide having a low melting point vary according to each technique, but it is preferably 0.5% by weight to 50% by weight with respect to the weight of the filler(s), 1% by weight to 40% by weight in another embodiment, and 2% by weight to 30% by weight in still another embodiment, or it is preferably 1% by weight to 20% by weight with respect to the weight of the formulation.

With respect to the type, formulation, content, and the like of other additives optionally used, the contents of the above-mentioned patent references disclosing rapidly disintegrating tablets in the buccal cavity are incorporated herein by reference.

In the case that the granular pharmaceutical composition of the present invention is contained in the rapidly disintegrating tablet in the buccal cavity, the granular pharmaceutical composition may be contained therein in an amount corresponding to 0.5% by weight to 90% by weight with respect to that of the rapidly disintegrating tablet in the buccal cavity, preferably 0.5% by weight to 80% by weight, and 1% by weight to 60% by weight in another embodiment.

Hereinafter the process of manufacturing the granular pharmaceutical composition of the present invention will be explained, but the present invention is not limited thereto.

The pharmaceutical composition of the present invention may be produced in accordance with a known method per se, such as pulverization, mixing, coating, granulation, sieving, drying, or the like.

A method for pulverization is not particularly limited with respect to an apparatus and procedures, so long as it is pharmaceutically acceptable. Examples of a pulverizer include a hammer mill, a ball mill, and a jet mill. The conditions for pulverization may be appropriately selected and are not particularly limited.

To obtain the granular pharmaceutical composition of the present invention, the core may be coated with a coating containing atorvastatin or a pharmaceutically acceptable salt thereof, the water-soluble polymer, and sodium laurylsulfate and/or polyoxyethylene hydrogenated castor oil. A particle consisting only of the drug may be used as the core containing the drug. Alternatively, a conventional technique may be used to prepare a particle consisting of the drug and an additive or two or more additives, in order to use as the core containing the drug. In this process of preparing the particle consisting of the drug and additive(s), for example, the drug and one or more appropriate fillers (such as crystalline cellulose, lactose, corn starch, or the like) may be mixed, together with a binder (such as hydroxypropyl cellulose or the like) if necessary, and granulated, sieved, and dried. Alternatively, an additive particle [for example, crystalline cellulose (particle) (sometimes referred to as microcrystalline cellulose), purified sucrose spheres, sucrose-starch spheres, or the like] as an appropriate core may be sprayed with a solution or dispersion liquid containing the drug and a binder.

As a method of coating the core with a coating containing atorvastatin or a pharmaceutically acceptable salt thereof, the water-soluble polymer, and sodium laurylsulfate and/or polyoxyethylene hydrogenated castor oil (hereinafter sometimes referred to as surfactant), any method capable of coating a granular pharmaceutical composition, such as a fluidized bed granulating and coating apparatus, a tumbling fluidized bed granulating and coating apparatus, a centrifugal tumbling granulating and coating apparatus, an agitation granulating apparatus, or the like, may be used. For example, in a fluidized bed granulating and side-spray coating apparatus with a side-spray, an appropriate amount of liquid containing a coating component may be sprayed using a spray gun, while the core particles containing the drug are fluidized by a warm air. The liquid containing a coating component may be prepared by dissolving or dispersing the essential component in a solvent such as water, ethanol, or methanol. These solvents may be used as an appropriate mixture.

In fluidized bed coating, for example, core particles are loaded into a fluidized bed granulating and coating apparatus, and coated by spraying a dispersion liquid containing atorvastatin or a pharmaceutically acceptable salt thereof, the surfactant, and the water-soluble polymer on the core particles, to prepare the particles.

When an agitation granulator is used, for example, the following procedures may be selected:
1) Atorvastatin or a pharmaceutically acceptable salt thereof, the surfactant, and the water-soluble polymer are loaded into an agitation granulator, and water is added or sprayed to prepare particles.
2) Atorvastatin or a pharmaceutically acceptable salt thereof and the surfactant are loaded into an agitation granulator, and an aqueous solution of the water-soluble polymer is added or sprayed to prepare particles.
3) Atorvastatin or a pharmaceutically acceptable salt thereof is loaded into an agitation granulator, and an aqueous solution containing the surfactant and the water-soluble polymer is added or sprayed to prepare particles.

When the tumbling fluidized bed granulating and coating apparatus is used, for example, core particles are loaded into the tumbling fluidized bed granulating and coating apparatus, and coated by spraying a dispersion liquid containing sodium laurylsulfate and/or polyoxyethylene hydrogenated castor oil, hypromellose, and atorvastatin or a pharmaceutically acceptable salt thereof to prepare particles.

A preferred spray rate for coating may vary according to a process or a scale thereof. At a 1 kg scale using the fluidized bed granulating and coating apparatus, a preferred spray rate is, for example, 2 g/min to 10 g/min, and 5 g/min to 10 g/min in another embodiment.

A preferred temperature of the drug-containing core particle to be coated is 15° C. to 60° C., 15° C. to 50° C. in another embodiment, and 15° C. to 45° C. in still another embodiment.

The granular pharmaceutical composition obtained by applying the drug-containing particles with a coating may be further dried, heated, or the like.

The particle size of the granular pharmaceutical composition of the present invention is not particularly limited, so long as the maximum length thereof is 2 mm or less. In the case that the granular pharmaceutical composition is contained in the rapidly disintegrating tablet in the buccal cavity, it is not particularly limited, so long as graininess like sands is not unpleasant in the administration, and the average particle size is preferably 350 μm or less, 1 μm to 350 μm in another embodiment, and 20 μm to 350 μm in still another embodiment.

The granulated product may be further dried, heated, or the like. A preferred temperature of the product is, for example, 30° C. to 70° C., and 40° C. to 70° C. in another embodiment.

The granular pharmaceutical composition of the present invention may be further coated with one or more pharmaceutical fillers.

The granular pharmaceutical composition of the present invention may be prepared by tabletting. Examples of the tabletting include a direct tabletting method in which drug-containing particles are mixed with an appropriate additive(s), and the mixture is compression-molded to obtain tablets; a method in which a composition obtained by a wet granulation (the granulation is carried out by spraying a mixture of drug-containing particles and additives with a binder liquid) or a melting granulation (the granulation is carried out by heating a mixture of drug-containing particles and an appropriate low-melting substance) is formed into tablets; and the like.

A rotary tabletting machine, a single punch tabletting machine, and the like may be used as a tabletting machine. A method as well as an apparatus is not particularly limited, so long as a compression-molded product can be pharmaceutically produced.

The use of sodium laurylsulfate and/or polyoxyethylene hydrogenated castor oil, and water-soluble polymer of the present invention is a use of sodium laurylsulfate and/or polyoxyethylene hydrogenated castor oil, and water-soluble polymer in the manufacture of a granular pharmaceutical composition for oral administration having a rapid dissolution of atorvastatin or a pharmaceutically acceptable salt thereof in the gastrointestinal tract. The explanation for the granular pharmaceutical composition of the present invention is cited as the detail description of the invention for the use.

### Examples

The present invention now will be further illustrated by, but is by no means limited to, the following Examples.

Atorvastatin calcium trihydrate was prepared by pulverizing crystalline Form I atorvastatin prepared in accordance with the method described in Examples of Japanese Patent No. 3296564 (WO97/03959), in order to use in the following Examples. Formulations in the following Examples are shown in Table 1 to Table 3.

TABLE 1

| | Examples (mg) | | | | | | | | | |
|---|---|---|---|---|---|---|---|---|---|---|
| | 1 | 2 | 3 | 4 | 5 | 6 | 7 | 8 | 9 | 10 |
| Crystalline | 21.6 | 19.5 | 17.4 | 21.6 | 19.5 | 18.4 | — | — | — | — |
| cellulose | | | | | | | | | | |
| (particle) | | | | | | | | | | |
| Magnesium | — | — | — | — | — | — | — | — | — | 6.2 |
| hydroxide | | | | | | | | | | |
| Atorvastatin | 10.8 | 10.8 | 10.8 | 10.8 | 10.8 | 10.8 | 10.8 | 10.8 | 10.8 | 10.8 |
| calcium | | | | | | | | | | |
| trihydrate | | | | | | | | | | |
| Sodium | 6.5 | 6.5 | 4.4 | 10.8 | 6.5 | 6.5 | 10.8 | 6.5 | — | 6.5 |
| laurylsulfate | | | | | | | | | | |
| Polyoxyethylene | — | — | — | — | — | — | — | — | 6.5 | — |
| hydrogenated | | | | | | | | | | |
| castor oil | | | | | | | | | | |
| Hypromellose | 4.3 | 2.2 | 2.2 | 2.2 | — | — | 2.2 | 4.3 | 4.3 | 4.3 |
| (TC-5E) | | | | | | | | | | |
| Hydroxypropyl | — | — | — | — | 2.2 | — | — | — | — | — |
| cellulose | | | | | | | | | | |
| Hypromellose | — | — | — | — | — | 1.1 | — | — | — | — |
| (TC-5R) | | | | | | | | | | |

TABLE 2

| | Examples (mg) | | | |
|---|---|---|---|---|
| | 11 | 12 | 13 | 14 |
| Crystalline cellulose (particle) | 21.6 | 19.5 | — | — |
| Atorvastatin calcium trihydrate | 10.8 | 10.8 | 10.8 | 10.8 |
| Sodium laurylsulfate | — | 6.5 | — | 10.8 |
| Polyoxyethylene hydrogenated | 6.5 | — | 10.8 | — |
| castor oil | | | | |
| Hypromellose (TC-5E) | 4.3 | — | 4.3 | 4.3 |
| Hypromellose (TC-5R) | — | 2.2 | — | — |

TABLE 3

| | | Examples (mg) | | |
|---|---|---|---|---|
| | | 15 | 16 | 17 |
| Core | Crystalline cellulose | 10.8 | 10.8 | 10.8 |
| | (particle) | | | |
| First layer | Atorvastatin calcium | 10.8 | 10.8 | 10.8 |
| | trihydrate | | | |
| | Sodium laurylsulfate | 6.5 | 6.5 | 6.5 |
| | Hypromellose (TC-5E) | 4.3 | 4.3 | 4.3 |
| Second layer | Sodium citrate hydrate | 5.6 | 5.6 | 5.6 |
| | Methyl cellulose | 4.9 | 4.9 | 4.9 |
| Third layer | Aminoalkylmethacrylate | 10.2 | 10.2 | 10.2 |
| | copolymer E | | | |
| | Talc | 5.8 | 5.8 | 5.8 |
| | Hypromellose (TC-5E) | 1.2 | 1.2 | 1.2 |
| Fourth layer | D-mannitol | 3.0 | 3.0 | 3.0 |
| Filler | D-mannitol | — | 214.6 | — |
| | Maltose syrup powder | — | 22.3 | — |
| | Lactose hydrate | — | — | 206.9 |
| Disintegrating | Low-substituted | — | — | 30.0 |
| agent | hydroxypropyl cellulose | | | |

### Example 1

To a solution prepared by dissolving 150.0 g of sodium laurylsulfate (Nikko Chemicals Co., Ltd., product name: NIKKOL SLS, the same compound was used in the following examples) and 100.0 g of hypromellose (Shin-Etsu Chemical Co., Ltd., product name: TC-5E, the same compound was used in the following examples, unless otherwise specified) in 2000.0 g of purified water, 250.0 g of atorvastatin calcium hydrate (Pfizer Inc., the same compound was used in the following examples) was added while stirring to prepare a dispersion liquid. The prepared dispersion liquid was sprayed on 500.0 g of crystalline cellulose (particle) (Asahi Kasei Chemicals Corporation, product name: CP-102Y, the same compound was used in the following examples) using a fluidized bed granulating apparatus (Glatt GmbH, product name: GPCG-1, the same apparatus was used in the following examples) to obtain a granular pharmaceutical composition of the present invention (Conditions for fluidized bed granulation: spray speed=7.0 g/min, air pressure of the spray=0.20 MPa). The average particle size of the obtained particles was 194 μm.

### Example 2

To a solution prepared by dissolving 150.0 g of sodium laurylsulfate and 50.0 g of hypromellose in 1800.0 g of purified water, 250.0 g of atorvastatin calcium hydrate was added while stirring to prepare a dispersion liquid. The prepared dispersion liquid was sprayed on 450.0 g of crystalline cellulose (particle) in a similar fashion as shown in Example 1 to obtain a granular pharmaceutical composition of the present invention. The average particle size of the obtained particles was 182 μm.

### Example 3

To a solution prepared by dissolving 125.0 g of sodium laurylsulfate and 62.5 g of hypromellose in 2000.0 g of purified water, 312.5 g of atorvastatin calcium hydrate was added while stirring to prepare a dispersion liquid. The prepared dispersion liquid was sprayed on 500.0 g of crystalline cellulose (particle) in a similar fashion as shown in Example 1 to obtain a granular pharmaceutical composition of the present invention.

### Example 4

To a solution prepared by dissolving 162.6 g of sodium laurylsulfate and 32.6 g of hypromellose in 1431.0 g of purified water, 162.6 g of atorvastatin calcium hydrate was added while stirring to prepare a dispersion liquid. The prepared dispersion liquid was sprayed on 325.2 g of crystalline cellulose (particle) using a fluidized bed granulating apparatus to obtain a granular pharmaceutical composition of the present invention (Conditions for fluidized bed granulation: spray speed=6.0 g/min, air pressure of the spray=0.20 MPa). The average particle size of the obtained particles was 178 μm.

### Example 5

To a solution prepared by dissolving 150.0 g of sodium laurylsulfate and 50.0 g of hydroxypropyl cellulose (Nippon Soda Co., Ltd., product name: NISSO HPC-SL) in 1800.0 g of purified water, 250.0 g of atorvastatin calcium hydrate was added while stirring to prepare a dispersion liquid. The prepared dispersion liquid was sprayed on 450.0 g of crystalline cellulose (particle) in a similar fashion as shown in Example 1 to obtain a granular pharmaceutical composition of the present invention. The average particle size of the obtained particles was 178 μm.

### Example 6

To a solution prepared by dissolving 156.0 g of sodium laurylsulfate and 26.0 g of hypromellose (Shin-Etsu Chemical Co., Ltd., product name: TC-5R) in 1800.0 g of purified water, 260.0 g of atorvastatin calcium hydrate was added while stirring to prepare a dispersion liquid. The prepared dispersion liquid was sprayed on 442.0 g of crystalline cellulose (particle) in a similar fashion as shown in Example 1 to obtain a granular pharmaceutical composition of the present invention. The average particle size of the obtained particles was 171 μm.

### Example 7

To a solution prepared by dissolving 10.8 g of sodium laurylsulfate and 2.2 g of hypromellose in 52.0 g of purified water, 10.8 g of atorvastatin calcium hydrate was added while stirring to prepare a dispersion liquid. The prepared dispersion liquid was dried at 40° C., and pulverized to prepare a granular pharmaceutical composition of the present invention.

### Example 8

To a solution prepared by dissolving 1.3 g of sodium laurylsulfate and 0.86 g of hypromellose in 17.3 g of purified water, 2.16 g of atorvastatin calcium hydrate was added while stirring to prepare a dispersion liquid. The prepared dispersion liquid was dried at 40° C., and pulverized to prepare a granular pharmaceutical composition of the present invention.

### Example 9

To a solution prepared by dissolving 1.3 g of polyoxyethylene hydrogenated castor oil (Nikko Chemicals Co., Ltd., product name: NIKKOLHCO-60) and 0.86 g of hypromellose in 17.3 g of purified water, 2.16 g of atorvastatin calcium hydrate was added while stirring to prepare a dispersion liquid. The prepared dispersion liquid was dried at 40° C., and pulverized to prepare a granular pharmaceutical composition of the present invention.

### Example 10

To a solution prepared by dissolving 420.0 g of sodium laurylsulfate and 280.0 g of hypromellose in 5600.0 g of purified water, 700.0 g of atorvastatin calcium hydrate was added while stirring to prepare a dispersion liquid. The prepared dispersion liquid was sprayed on 400.0 g of magnesium hydroxide (Tomita Pharmaceutical Co., Ltd.) in a similar fashion as shown in Example 1 to obtain a granular pharmaceutical composition of the present invention. The average particle size of the obtained particles was 201 μm.

### Example 11

To a solution prepared by dissolving 150.0 g of polyoxyethylene hydrogenated castor oil and 100.0 g of hypromellose in 2000.0 g of purified water, 250.0 g of atorvastatin calcium hydrate was added while stirring to prepare a dispersion liquid. The prepared dispersion liquid was sprayed on 500.0 g of crystalline cellulose (particle) using a fluidized bed granulating apparatus to obtain a granular pharmaceutical composition of the present invention (Conditions for fluidized bed granulation: spray speed=6.8 g/min, air pressure of the spray=0.25 MPa).

### Example 12

To a solution prepared by dissolving 150.0 g of sodium laurylsulfate and 50.0 g of hypromellose (Shin-Etsu Chemical Co., Ltd., product name: TC-5R) in 1800.0 g of purified water, 250.0 g of atorvastatin calcium hydrate was added while stirring to prepare a dispersion liquid. The prepared dispersion liquid was sprayed on 450.0 g of crystalline cellulose (particle) using a fluidized bed granulating apparatus to obtain a granular pharmaceutical composition of the present invention (Conditions for fluidized bed granulation: spray speed=7.0 g/min, air pressure of the spray=0.20 MPa). The average particle size of the obtained particles was 177 μm.

### Example 13

To a solution prepared by dissolving 5.0 g of polyoxyethylene hydrogenated castor oil and 2.0 g of hypromellose in 68.0 g of purified water, 5.0 g of atorvastatin calcium hydrate was added while stirring to prepare a dispersion liquid. The prepared dispersion liquid was dried at 40° C., and pulverized to prepare a granular pharmaceutical composition of the present invention.

### Example 14

To a solution prepared by dissolving 5.0 g of sodium laurylsulfate and 2.0 g of hypromellose in 68.0 g of purified water, 5.0 g of atorvastatin calcium hydrate was added while stirring to prepare a dispersion liquid. The prepared dispersion liquid was dried at 40° C., and pulverized to prepare a granular pharmaceutical composition of the present invention.

### Example 15

### (1) Preparation of First Layer

To a solution prepared by dissolving 180.0 g of sodium laurylsulfate and 120.0 g of hypromellose in 2400.0 g of purified water, 300.0 g of atorvastatin calcium hydrate was added while stirring to prepare a dispersion liquid. The prepared dispersion liquid was sprayed on 300.0 g of crystalline cellulose (particle) using a fluidized bed granulating apparatus to obtain a granular pharmaceutical composition of the present invention (Conditions for fluidized bed granulation: spray speed=7.0 g/min, air pressure of the spray=0.25 MPa).

### (2) Preparation of Second Layer

A solution prepared by dissolving 51.3 g of sodium citrate hydrate (Wako Pure Chemical Industries, Ltd.) and 45.0 g of methyl cellulose (Shin-Etsu Chemical Co., Ltd., product name: METOLOSE SM-4) in 1189.5 g of purified water was sprayed on 300.0 g of the particles coated with the first layer using a fluidized bed granulating apparatus to prepare particles coated with the second layer (Conditions for fluidized bed granulation: spray speed=6.0 g/min, air pressure of the spray=0.25 MPa).

### (3) Preparation of Third Layer

To a solution prepared by dissolving 71.2 g of aminoalkylmethacrylate copolymer E (Evonik Degussa GmbH, Product name: Eudragit E100) in 1824.0 g of methanol, 40.7 g of talc (Kihara Kasei Co., Ltd., Product name: Highfiller #17) was dispersed, and a solution prepared by dissolving 8.1 g of hypromellose in 456.0 g of purified water was added. The resulting dispersion was sprayed on 300.0 g of the particles coated with the second layer using a fluidized bed granulating apparatus to prepare particles coated with the third layer (Conditions for fluidized bed granulation: spray speed=7.0 g/min, air pressure of the spray=0.2 MPa).

### (4) Preparation of Fourth Layer

A solution prepared by dissolving 20.0 g of D-mannitol (ROQUETTE, product name: PEARLITOL 50C, the same compound was used in the following examples) in 180.0 g of purified water was sprayed on 400.0 g of the particles coated with the third layer using a fluidized bed granulating apparatus to prepare particles coated with the fourth layer (Conditions for fluidized bed granulation: spray speed=5.1 g/min, air pressure of the spray=0.18 MPa).

### Example 16

A mixture of 557.8 g of D-mannitol and 6.5 g of maltose syrup powder (Hayashibara Shoji, Inc., Sunmalt S) was granulated with 258 g of a solution of maltose syrup powder (containing 51.6 g of maltose syrup powder) using a fluidized bed granulating apparatus to prepare a granulated product for a rapidly disintegrating tablet in the buccal cavity. A mixture of 236.9 mg of the resulting granulated product and 63.1 mg of the particles coated with the fourth layer prepared in Example 15 was filled in a die having a diameter of 9.5 mm, and tabletted using an autograph (Shimadzu, AGS-20KNG, the same apparatus was used in the following examples) to prepare a rapidly disintegrating tablet in the buccal cavity containing a granular pharmaceutical composition of the present invention.

A mixture prepared by mixing 206.9 mg of lactose hydrate (Freund Corporation, product name: Dilactose S) and 30.0 mg of low-substituted hydroxypropyl cellulose (Shin-Etsu Chemical Co., Ltd., product name: L-HPC LH-21) with 63.1 mg of the particles coated with the fourth layer prepared in Example 15 was filled in a die having a diameter of 9.5 mm, and tabletted using an autograph to prepare a tablet containing a granular pharmaceutical composition of the present invention.

Formulations in the following Comparative Examples are shown in Table 4 and Table 5.

TABLE 4

| | Comparative Examples | | | | |
|---|---|---|---|---|---|
| | 1 | 2 | 3 | 4 | 5 |
| Crystalline cellulose | 13.0 | — | — | — | — |
| (particle) | | | | | |
| Atorvastatin calcium | 10.8 | 10.8 | 10.8 | 10.8 | 10.8 |
| trihydrate | | | | | |
| Crospovidone | — | 5.4 | — | — | — |
| Polysorbate 80 | — | — | 6.5 | — | — |
| Sugar fatty acid ester | — | — | — | 6.5 | — |
| Dioctyl sodium | — | — | — | — | 6.5 |
| sulfosuccinate | | | | | |
| Hypromellose (TC-5E) | 2.2 | 2.2 | 4.3 | 4.3 | 4.3 |

TABLE 5

| | Comparative | | |
|---|---|---|---|
| | Examples (mg) | | |
| | 6 | 7 | 8 |
| Atorvastatin calcium trihydrate | 10.8 | 10.8 | 10.8 |
| Polyethylene glycol monostearate | 6.5 | 10.8 | — |
| Polysorbate 80 | — | — | 10.8 |
| Hypromellose (TC-5E) | 4.3 | 4.3 | 4.3 |

### Comparative Example 1

To a solution prepared by dissolving 84.1 g of hypromellose in 2016.0 g of purified water, 419.9 g of atorvastatin calcium hydrate was added while stirring to prepare a dispersion liquid. The prepared dispersion liquid was sprayed on 504.0 g of crystalline cellulose (particle) using a fluidized bed granulating apparatus to obtain a granular pharmaceutical composition for comparison (Conditions for fluidized bed granulation: spray speed=7.0 g/min, air pressure of the spray=0.20 MPa). The average particle size of the obtained particles was 202 μm.

### Comparative Example 2

To a solution prepared by dissolving 2.2 g of hypromellose in 52.0 g of purified water, 5.4 g of crospovidone (BASF, product name: KollidonCL) and 10.8 g of atorvastatin calcium hydrate were added while stirring to prepare a dispersion liquid. The prepared dispersion liquid was dried at 40° C., and pulverized to prepare a granular pharmaceutical composition for comparison.

### Comparative Example 3

To a solution prepared by dissolving 1.3 g of polysorbate 80 (Merck, product name: Tween80) and 0.86 g of hypromellose in 17.3 g of purified water, 2.16 g of atorvastatin calcium hydrate was added while stirring to prepare a dispersion liquid. The prepared dispersion liquid was dried at 40° C., and pulverized to prepare a granular pharmaceutical composition for comparison.

### Comparative Example 4

To a solution prepared by dissolving 1.3 g of sugar fatty acid ester (Dai-ichi Kogyo Seiyaku Co., Ltd., product name: DK ester SS) and 0.86 g of hypromellose in 17.3 g of purified water, 2.16 g of atorvastatin calcium hydrate was added while stirring to prepare a dispersion liquid. The prepared dispersion liquid was dried at 40° C., and pulverized to prepare a granular pharmaceutical composition for comparison.

### Comparative Example 5

To a solution prepared by dissolving 1.3 g of dioctyl sodium sulfosuccinate (CYTEC Industries Inc., product name: DOCUSATE SODIUM U.S.P.) and 0.86 g of hypromellose in 17.3 g of purified water, 2.16 g of atorvastatin calcium hydrate was added while stirring to prepare a dispersion liquid. The prepared dispersion liquid was dried at 40° C., and pulverized to prepare a granular pharmaceutical composition for comparison.

### Comparative Example 6

To a solution prepared by dissolving 3.0 g of polyethylene glycol monostearate (Nikko Chemicals Co., Ltd., product name: NIKKOL MYS-40MV, the same compound was used in the following examples) and 2.0 g of hypromellose in 56.7 g of purified water, 5.0 g of atorvastatin calcium hydrate was added while stirring to prepare a dispersion liquid. The prepared dispersion liquid was dried at 40° C., and pulverized to prepare a granular pharmaceutical composition for comparison.

### Comparative Example 7

To a solution prepared by dissolving 5.0 g of polyethylene glycol monostearate and 2.0 g of hypromellose in 68.0 g of purified water, 5.0 g of atorvastatin calcium hydrate was added while stirring to prepare a dispersion liquid. The prepared dispersion liquid was dried at 40° C., and pulverized to prepare a granular pharmaceutical composition for comparison.

### Comparative Example 8

To a solution prepared by dissolving 5.0 g of polysorbate 80 and 2.0 g of hypromellose in 68.0 g of purified water, 5.0 g of atorvastatin calcium hydrate was added while stirring to prepare a dispersion liquid. The prepared dispersion liquid was dried at 40° C., and pulverized to prepare a granular pharmaceutical composition for comparison.

### Experimental Example

With respect to the granular pharmaceutical compositions, the rapidly disintegrating tablet in the buccal cavity, and the tablet prepared in Examples 1 to 17 and Comparative Examples 1 to 8, particles containing 10 mg of the drug were weighed out, and the dissolution rate for 15 minutes (D15min) was determined in accordance with a dissolution test, method 2 described in the Japanese Pharmacopoeia, using 900 mL of the first fluid (JP1) described in this dissolution test of the Japanese Pharmacopoeia, or 900 mL of a solution prepared by dissolving 0.05% by weight of sodium laurylsulfate in the first fluid (JP1). The atorvastatin formulation [Pfizer Inc., Lipitor (registered trademark)] was used as a control.

The results of the dissolution test are shown in Tables 6 to 8. It was clarified from the results of Examples that the granular pharmaceutical composition of the present invention exhibited a rapid dispersibility or dissolution the same as or superior to that of the control formulation in the first fluid (JP1) described in this dissolution test of the Japanese Pharmacopoeia, by using a specific surfactant and a water-soluble polymer.

As shown in Examples, addition of sodium laurylsulfate or polyoxyethylene hydrogenated castor oil resulted in a rapid dissolution even after 15 minutes from the beginning of the dissolution. It was found from Comparative Examples 2 to 8 that addition of sodium laurylsulfate or polyoxyethylene hydrogenated castor oil showed a more rapid dissolution in comparison with commonly used surfactants, and advantageous effects of sodium laurylsulfate and polyoxyethylene hydrogenated castor oil were shown.

In general, a surfactant is added in a small amount from the viewpoint of productivity. Despite a large amount of surfactant was added in Examples 16 and 17, drug-containing particles could be coated with a coating substance. In addition, the compositions of the present invention showed a rapid dissolution, even after coating with a coating substance or tabletting.

TABLE 6

Dissolution rate (%)

| | Control | Example 1 | Example 2 | Example 3 | Example 4 |
|---|---|---|---|---|---|
| D₁₅ₘᵢₙ | 48.0 | 96.0 | 90.3 | 57.9 | 100.0 |
| | | | | | Example |
| | Example 5 | Example 6 | Example 10 | Example 11 | 12 |
| D₁₅ₘᵢₙ | 88.1 | 86.3 | 69.1 | 85.4 | 88.1 |

| | | | | Comparative |
|---|---|---|---|---|
| | Example 15 | Example 16 | Example 17 | Example 1 |
| D₁₅ₘᵢₙ | 85.8 | 78.2 | 82.9 | 12.4 |

JP1 (900 mL) was used as a test liquid, and the paddle rotation speed was 100 rpm.

TABLE 7

Dissolution rate (%)

| | Example 7 | Comparative Example 2 |
|---|---|---|
| D_{15 min} | 98.9 | 38.8 |

The solution (900 mL) prepared by dissolving 0.05% by weight of sodium laurylsulfate in JP1 was used as a test liquid, and the paddle rotation speed was 75 rpm.

TABLE 8

| | Dissolution rate (%) | | | | |
|---|---|---|---|---|---|
| | | | | | Compar- |
| | | | | | ative |
| | Example 8 | Example 9 | Example 13 | Example 14 | Example 3 |
| D₁₅ₘᵢₙ | 69.6 | 77.6 | 93.6 | 83.6 | 28.5 |

| | | | Compar- | Compar- | Compar- |
|---|---|---|---|---|---|
| | Comparative | Comparative | ative | ative | ative |
| | Example 4 | Example 5 | Example 6 | Example 7 | Example 8 |
| D₁₅ₘᵢₙ | 18.6 | 7.1 | 26.1 | 45.2 | 55.3 |

JP1 (900 mL) was used as a test liquid, and the paddle rotation speed was 50 rpm.

### INDUSTRIAL APPLICABILITY

The granular pharmaceutical composition of the present invention shows a drug dispersibility or dissolution the same as or superior to that of a solid formulation containing atorvastatin or a pharmaceutically acceptable salt thereof, which is now provided to the medical field, and is useful as a pharmaceutical composition showing a rapid dispersibility or dissolution in the gastrointestinal tract, even if the unpleasant taste of the drug in the buccal cavity is masked.

As above, the present invention was explained with reference to particular embodiments, but modifications and improvements obvious to those skilled in the art are included in the scope of the present invention.

1. A granular pharmaceutical composition for oral administration, comprising (1) atorvastatin or a pharmaceutically acceptable salt thereof, (2) a surfactant selected from the group consisting of sodium laurylsulfate and polyoxyethylene hydrogenated castor oil, and (3) a water-soluble polymer.

2. The granular pharmaceutical composition for oral administration according to claim 1, wherein an amount of the surfactant is 30% by weight to 200% by weight with respect to an amount of atorvastatin or a pharmaceutically acceptable salt thereof.

3. The granular pharmaceutical composition for oral administration according to claim 1, wherein an amount of the surfactant is 40% by weight to 100% by weight with respect to an amount of atorvastatin or a pharmaceutically acceptable salt thereof

4. The granular pharmaceutical composition for oral administration according to claim 1, wherein the surfactant is sodium laurylsulfate.

5. The granular pharmaceutical composition for oral administration according to claim 1, wherein the water-soluble polymer has a viscosity of approximately 2 mPa·s to approximately 100 mPa·s.

6. The granular pharmaceutical composition for oral administration according to claim 1, wherein the water-soluble polymer is one member, or two or more members selected from the group consisting of hydroxypropylmethyl cellulose, hydroxypropyl cellulose, methyl methacrylate-butyl methacrylate-dimethylaminoethyl methacrylate copolymer coexistent with an acidic substance in an amount which neutralizes 10% or more of basic groups of the copolymer, povidone, and methyl cellulose.

7. The granular pharmaceutical composition for oral administration according to claim 1, wherein an amount of the water-soluble polymer is 5% by weight to 100% by weight with respect to an amount of atorvastatin or a pharmaceutically acceptable salt thereof.

8. The granular pharmaceutical composition for oral administration according to claim 1, wherein an amount of the water-soluble polymer is 10% by weight to 40% by weight with respect to an amount of atorvastatin or a pharmaceutically acceptable salt thereof.

9. The granular pharmaceutical composition for oral administration according to claim 1, which contains a core.

10. The granular pharmaceutical composition for oral administration according to claim 9, wherein the core is coated with a coating comprising (1) atorvastatin or a pharmaceutically acceptable salt thereof, (2) the surfactant selected from the group consisting of sodium laurylsulfate and polyoxyethylene hydrogenated castor oil, and (3) the water-soluble polymer.

11. The granular pharmaceutical composition for oral administration according to claim 9, wherein the core is one member, or two or more members selected from the group consisting of crystalline cellulose, purified sucrose spheres, D-mannitol, magnesium hydroxide, lactose-crystalline cellulose spheres, and sucrose-starch spheres.

12. The granular pharmaceutical composition for oral administration according to claim 1, wherein atorvastatin or a pharmaceutically acceptable salt thereof exists as a crystal.

13. The granular pharmaceutical composition for oral administration according to claim 1, wherein atorvastatin or a pharmaceutically acceptable salt thereof exists as a type I crystal.

14. The granular pharmaceutical composition for oral administration according to claim 1, wherein atorvastatin or a pharmaceutically acceptable salt thereof is atorvastatin calcium.

15. A process of manufacturing a granular pharmaceutical composition for oral administration, comprising mixing atorvastatin or a pharmaceutically acceptable salt thereof with a water-soluble polymer and a surfactant selected from the group consisting of sodium laurylsulfate and polyoxyethylene hydrogenated castor oil.

16. The process according to claim 15, wherein an amount of the surfactant is 30% by weight to 200% by weight with respect to an amount of atorvastatin or a pharmaceutically acceptable salt thereof.

17. The process according to claim 15, wherein an amount of the surfactant is 40% by weight to 100% by weight with respect to an amount of atorvastatin or a pharmaceutically acceptable salt thereof.

18. The process according to claim 15, wherein the surfactant is sodium laurylsulfate.

19. The process according to claim 15, wherein the water-soluble polymer has a viscosity of approximately 2 mPa·s to approximately 100 mPa·s.

20. The process according to claim 15, wherein the water-soluble polymer is one member, or two or more of members selected from the group consisting of hydroxypropylmethyl cellulose, hydroxypropyl cellulose, methyl methacrylate-butyl methacrylate-dimethylaminoethyl methacrylate copolymer coexistent with an acidic substance in an amount which neutralizes 10% or more of basic groups of the copolymer, povidone, and methyl cellulose.

21. The process according to claim 15, wherein an amount of the water-soluble polymer is 5% by weight to 100% by weight with respect to an amount of atorvastatin or a pharmaceutically acceptable salt thereof.

22. The process according to claim 15, wherein an amount of the water-soluble polymer is 10% by weight to 40% by weight with respect to an amount of atorvastatin or a pharmaceutically acceptable salt thereof.

23. The process according to claim 15, which contains a core.

24. The process according to claim 23, wherein the core is coated with a coating comprising (1) atorvastatin or a pharmaceutically acceptable salt thereof, (2) the surfactant selected from the group consisting of sodium laurylsulfate and polyoxyethylene hydrogenated castor oil, and (3) the water-soluble polymer.

25. The process according to claim 23, wherein the core is one member, or two or more members selected from the group consisting of crystalline cellulose, purified sucrose spheres, D-mannitol, magnesium hydroxide, lactose-crystalline cellulose spheres, and sucrose-starch spheres.

26. The process according to claim 15, wherein atorvastatin or a pharmaceutically acceptable salt thereof is atorvastatin calcium.

27. (canceled)
